(19) United States
(12) Patent Application Publication
(10) Pub. No.: US 2008/0275328 A1
(43) Pub. Date: Nov. 6, 2008

(54) SENSING BODY FUNCTIONS

(76) Inventor: David Paul Jones, Buckinghamshire (GB)

Correspondence Address: RENNER, OTTO, BOISSELLE & SKLAR, LLP, Nineteenth Floor, 1621 Euclid Avenue, Cleveland, OH, 44115-2191, US

(21) Appl. No.: 11/964,962

(22) Filed: Dec. 27, 2007

Related U.S. Application Data
(63) Continuation of application No. PCT/GB2006/050169, filed on Jun. 27, 2006.

(30) Foreign Application Priority Data
Jun. 27, 2005 (GB) 0512946.5

(51) Int. Cl. A61B 5/05

(52) U.S. Cl. 600/407

(57) ABSTRACT

A body function sensor (1, 2, 3) for detecting breathing or heart beat or the like has an RF source (5) driving a transmit antenna (20) to create a low frequency (LF) electromagnetic detection field at a location to be occupied by an animal body. A receive antenna (21) is arranged to drive an analyser means (23, 13, 15,) to produce a signal representing the difference between the transmitted and detected signals detect a change in the field caused by the breathing or heart beat of an animal body in the location. A control means (16) controls the frequency of the RF source (5) to ensure resonance, and also controls the analyser means (23, 13), a display monitor (17) and an alarm. A memory (18) records movements of the subject, breathing patterns, pulse rate and posture of the body.

### RELATED APPLICATIONS

This application is a continuation of International Application No. PCT/GB2006/050169 filed on Jun. 27, 2006, which is hereby incorporated herein by reference in its entirety.

### FIELD OF THE INVENTION

The invention relates to the detection of certain body functions indicative of the physical state of an individual, human or animal and also to detecting presence at or absence from a location. The invention provides both a body function detector and a method of monitoring a body function of the individual at a location. The invention is particularly, but not exclusively, useful for detecting and monitoring physiological parameters of a patient, such as pulse, breathing, breathing patterns, and body posture whilst the patient is in a chair or in bed, and for detecting and monitoring whether the patient has left the bed or chair.

### BACKGROUND OF THE INVENTION

Throughout this specification the term “low frequency” (“LF”) has the internationally-accepted meaning of 30 kHz to 300 kHz.

In our International Patent Application WO 03/065324 we describe a method and apparatus for detecting the presence of an individual, for instance an intruder in a detection zone, and for determining the direction of movement of the intruder within that zone or the direction of movement towards/away from the zone. This method and apparatus is useful, for instance, for security, safety, rescue, access control, and energy management. Such method and apparatus use a low frequency (30-300 kHz) radio field established between two antennae. The intrusion of an individual, or other body, into the field causes a phase change in the field. This phase change is detected and, if necessary, corroborated, and an output generated in response to the detection.

U.S. Pat. No. 4,085,740 teaches a method for measuring physiological parameters such as pulse rate and respiration without physically connecting electrodes or other sensors to the body. A beam of phased microwave energy is directed towards the body of a person at a region thereon which undergoes physical displacement corresponding to variations in the parameter being measured. The phase relationship of the energy reflected from the body is compared with that of the transmitted energy to determine the extent of physical movement of the body region as affected by the parameter being measured. This disclosure teaches the use of a beam operating frequency of around 10,000 MHz that is sufficiently powerful as to be reflected by the body region.

### SUMMARY OF THE INVENTION

According to a first aspect of the invention a body function sensor, that is arranged to detect a body function of an animal body by detecting any phase difference between electromagnetic energy to which the animal body is exposed and electromagnetic energy received by an analyser means, is arranged to create a low frequency (LF) electromagnetic field at a location where the animal body is to be positioned and to detect any phase changes in this electromagnetic field, and has a control means to adjust the frequency of the electromagnetic field until it is resonant, and the analyser means is arranged to assess minor phase changes in the electromagnetic field as an indication of the body function.

In this manner, body functions (for instance heart rate, breathing patterns, coughing, etc.) may be sensed at a monitoring location without any need to connect electrodes or other sensors to the body and without any need to. transmit energetic beams at the body. The use of a low frequency (LF) field avoids any need to direct any beam at the body and does not compromise the safety of long-term environment of the body.

The analyser means is preferably arranged to determine the presence or movement of the animal body at the location by detecting a change in resonant frequency caused by the presence or movement of the animal body.

The analyser means is preferably arranged to determine at least one body function of the animal body by detecting changes in the phase of the electromagnetic field caused by the movement of the animal body at the location.

A variable frequency waveform generator may have its frequency controlled by the control means and be arranged to transmit its output through a transmit means to an antenna for transmitting the low frequency (LF) electromagnetic field at the location. The antenna may include a single conductor positioned at the location and also serving to transmit a received signal to an analyser means. Preferably a receive antenna is provided to receive the electromagnetic field and to convey, it to the analysis means. In this case, the antenna and the receive antenna include preferably separate conductors positioned at the location. Preferably these separate conductors are intercalated.

The variable frequency waveform generator may include a source of LF radio-frequency radiation and may be connected to the transmit antenna.

The analyser means may include an Analogue/Digital converter to receive a signal representative of the electromagnetic field, and a Fast Fourier Transformer or its electronic equivalent, to determine the difference in phase between the transmitted LF signal and a received LF signal changed by said body function.

The analyser means may include a filter to filter the difference in phase, and a monitor/display means operated by the control means to monitor/display the results of the sensor.

A memory is preferably provided to record the history of phase changes.

According to another aspect of the invention, a method of monitoring a body function of an animal body at a location, comprises creating a low frequency (LF) electromagnetic field at the location, adjusting the frequency of the electromagnetic field until it is resonant, assessing phase changes in the electromagnetic field, and analysing such phase changes to monitor the body function.

The method preferably uses a change in the frequency to indicate the presence or absence of the animal body at the location. In the case where the animal body is a human being, the method preferably includes by using either a bed or a chair as the location.

The method preferably includes recording changes in the frequency to provide a history of the times when the human being was in the bed or the chair.

The method preferably includes using the analysis of the phase changes to provide a real-time assessment of the well-being of the human being.

The method preferably includes recording phase changes to provide a history of the monitored body function.

The method may include operating an alarm whenever the human being is absent from the bed or chair, or the well-being of the human being is compromised.

The waveform generator Is preferably arranged to transmit at frequencies having low background noise.

### BRIEF DESCRIPTION OF THE DRAWINGS

The invention is now described, by way of example only, with reference to the accompanying drawings, in which:

FIG. 1 is a block diagram of a first form of body function sensor;

FIG. 2 is a block diagram of a preferred form of body function sensor;

FIG. 3 is a diagrammatic plan view of intercalated conductors arranged on a bed;

FIG. 4 is a graph showing frequency change against time and illustrating the significant frequency change as a patient get out of bed;

FIG. 5 is a graph showing phase change against time and illustrating the change in phase as a patient changes posture in bed, and

FIG. 6 is a set of graphs showing phase change against time and illustrating analysis of respiration.

### DETAILED DESCRIPTION OF THE ILLUSTRATED EMBODIMENTS

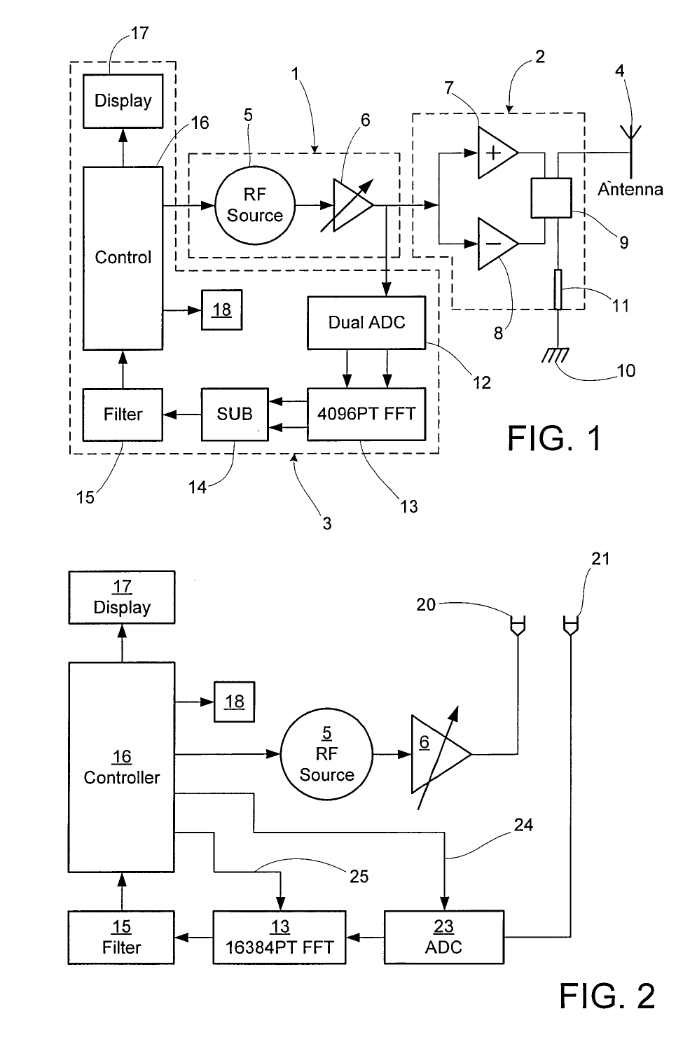

With reference to FIG. 1, a body function sensor comprises a generator means 1 to produce a variable source of low frequency (LF) signals in the range 50 to 300 kHz, a transmit means 2 coupling the LF signals to an antenna 4, and an analyser means 3 to sense the received LF signals and compare them to those originally transmitted, thereby producing an output representative of the breathing or other body function of the subject being monitored.

The generator means 1 consists of an RF source 5 connected to a variable gain amplifier 6, whose output passes to both the transmit means 2 and the analyser means 3. The transmit means 2 contains a pair of amplifiers 7 and 8 of opposite polarities which each receive the LF signal and couple it via transformer 9 to produce a balanced feed to the receive/transmit antenna 4. The secondary side of the transformer 9 is coupled to ground 10 via a resistor 11. A junction between the resistor 11 and the secondary winding of the transformer 9 is coupled to an input of the analyser means 3.

The receive/transmit antenna establishes a static, localised electromagnetic field in a location where the subject is to be monitored. The LF signal radiated by the transmit/receive antenna 4 is of very low power, some 100 to 1000 times lower than accepted continuous safe limits. It is also of low duty ratio as opposed to continuous wave radiation. There is therefore absolutely negligible risk to the subject being monitored, even where the subject is a child or a small animal.

The output of amplifier 6 is supplied to one channel of an A/D converter 12 to act as a reference signal. The other input to the A/D converter is taken from the transmit means 2 as already mentioned. The A/D converter produces two digital signals which are respectively fed to a Fast Fourier Transform (FFT) circuit 13, whose two outputs are passed to a subtracter 14. The net effect is effectively to generate a difference signal between the transmitted and detected LF signals. The apparatus can thereby be initialised and extraneous LF radiation eliminated from the detection.

When the subject is at the location, movement of the subject's lungs and/or the effect of the subject's pulse, causes disturbances to the static low frequency electromagnetic field radiated by the transmit/receive antenna 4. These disturbances are detected in the sensor means 3 by measuring the phase difference between the transmitted and received signals.

After passing through the filter 15, the resultant signal is passed to a control means 16 which converts the received signal into a form suitable for use in a display monitor 17. The control means 16 also feeds a signal back into the generator means 1 to control the frequency of the LF signal, or the combination of frequencies if the RF source 5 is a multi-frequency generator. A memory 18 is provided to record against time, the changes in the frequency and the analysed phase changes.

Certain frequencies within the LF bandwidth will offer greater discrimination of body functions, such as breathing patterns, than other frequencies. The control means 16 is therefore adjustable so as to select preferred frequencies. The control means 16 is arranged to adjust the frequency of the RF source 5 to ensure that the electromagnetic field is resonant. The electronic circuits and components necessary to achieve this are well-known in the art and, as they do not form part of the present invention, are not herein described.

The basic principle of operation of the sensor is the modification of the LF field by the presence of a body. A human or animal body exhibits values of both conductance and capacitance when intruding into this LF field, through the parameters of conductivity and permittivity. Whilst the exact scientific analysis is currently uncertain, such an intrusive body appears to act as a dielectric. We have found that, at the lower end of the preferred frequency range, the intrusive body acts principally as a conductor of the LF signals, with some resistive loss, but it also displays a capacitive component. Below 100 kHz the capacitive element appears to be typically less than 10% of the impedance of the body, but is higher above 100 kHz.

A set up signal is used to cause the control means 16 to apply a control signal to set the frequency range of the RF source 5 to comply with frequency regulatory issues in the vicinity of the sensor. The control means 16 uses the control signal not only to control the frequency of the RF source 5, but also amplitude and modulation. The control means 16 also transmits a further control signal to set the gain of the amplifier 6.

During the set-up routine of the sensor, and without the presence of a subject in the LF field, the control means 16 performs a frequency scan of the available frequencies within the 30 kHz to 300 kHz (preferably up to 200 kHz) spectrum to determine the background noise environment and to identify those channels that exhibit least noise characteristics.

In operation the body function sensor 1, 2, 3 is set by the control means 16 which alters the frequency of the RF source until the electromagnetic field is resonant in the absence of any animal body in the location. When an animal body arrives at the location, the electromagnetic field is disrupted, and loses resonance, typically up to 50 kHz. This loss of resonance is detected by the control means 16 which alters the frequency of the electromagnetic field to restore resonance. Once occupancy of the location has been determined, the resonance will change dependent on the movement and attitude of the animal body, typically within a band of +/−6 kHz. The control means 16 is arranged to adjust the frequency continually to maintain resonance. These variations in the resonant frequency are used to assess and monitor the movement and change of attitude of the animal body whilst at the location. Irrespective of any change in the resonant frequency, the analyser means 3 detects changes in the phase of the electromagnetic field caused by minor movements of the animal body at the location and analyses these as body functions such as heart rate and respiration rate and pattern.

The transmit/receive antenna 4 is preferably implemented as a single conductor which extends from the transformer 9 to the location where the animal body is to be monitored. In the case where the animal body is a human being the end of this single conductor remote from the transformer 9 may be embedded in a flexible substrate positioned on the upper surface of a bed or a chair in a similar manner to an electric pre-heating under-blanket. This ensures that the electromagnetic field is generated in close proximity to the subject. The ground plane 10 can be provided by the upper surface of the bed or its frame. Alternatively, the ground plane 10 may be provided by a planar conductive layer placed separately on the upper surface of the bed or chair, or by a planar layer built into the blanket. The ground plane can be effectively constituted by a mesh construction in which the mesh spacing is much less than a half wavelength at the operating frequency, e.g. much less than 1250 m. A rectangular metal bed frame, for example, may provide a satisfactory ground plane provided it is a closed loop.

It is not always necessary to provide a ground plane as such, provided there is a ground potential to which the circuitry can be referenced. The term “ground plane” therefore encompasses both alternatives.

FIG. 2 illustrates a preferred form of body function sensor that has several features similar to those described with reference to FIG. 1. Accordingly the same reference numerals are used to denote equivalent components, only the points of difference being described in detail.

The body function sensor of FIG. 2 has a separate transmit antenna 20 driven by the output from the variable gain amplifier 6, and a separate receive antenna 21 which feeds an analogue/digital converter 23. This simplifies the system shown in FIG. 1 by omitting its transmit means 2. The control means 16 has additional outputs 24 and 25 respectively connected to the analogue/digital converter 23 and to the Fast Fourier Transform circuit 13.

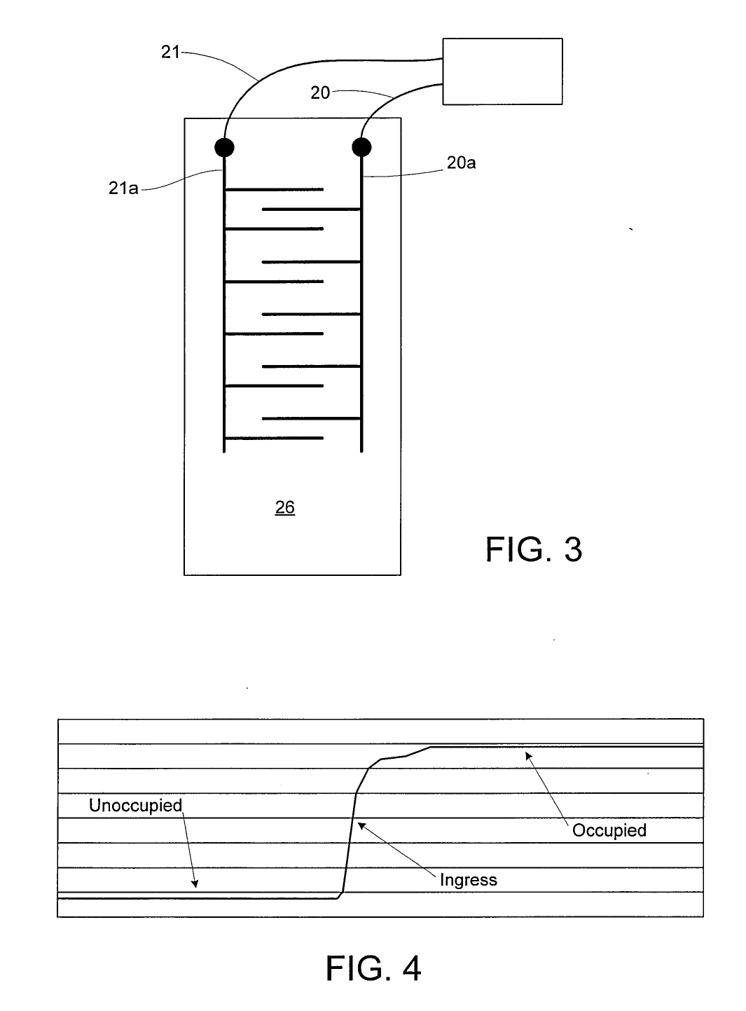

FIG. 3 diagrammatically depicts the arrangement of an end portion 20a of the transmit antenna 20, and an end portion 21a of the receive antenna 21, on a mattress 26 supported by an unshown bed. The end portions 20a and 21a are formed from conductive strips shaped as an intercalated ladder arrangement to ensure good continuity. It will be noted that the positioning and respective spacing of the end portions 20a and 21a ensure that a patient lying on the bed will always be in close proximity. The end portions 20a and 21a can be arranged either on top or underneath the mattress 26 and do not need to be in direct contact with the patient's body. As the patient's body moves through respiration, heart beat, normal turning, and irregular movements such as fits or seizures, the body functions and the pattern of movement are all detected, assessed and shown on the display monitor 17 and recorded as desired in the memory 18. Similarly major movements such as falling, or getting, out of the bed are detected, monitored and recorded.

FIG. 4 is a graph of frequency against time, running from the left towards the right, first showing an empty bed, ingress of a patient into the bed with typical movements as the patient adjusts position, followed by uninterrupted presence in the bed.

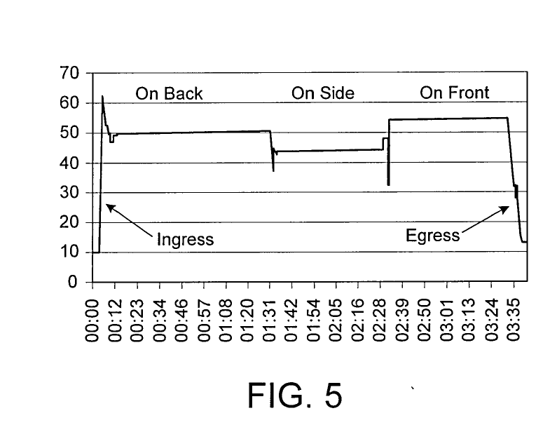

FIG. 5 is a graph of phase against time, running from left to right and monitoring respiration. As can readily be seen, air ingress is associated with a sharp increase in detected value, rising from 10 to over 60 in a fraction of a second. When the subject is on his back, the detected value stabilises at around 50. When on his side, the value settles at 45 and when on his front at 55, or thereabouts. Finally, at egress, the value falls rapidly back to around 10 once again.

Clearly, not only can the system detect whether or not a subject is actually breathing, it can detect the posture of the subject. The invention therefore detects breathing patterns and can either be used to detect the subject's posture or can be seen to be unaffected by posture, in that readings of 45 to 55 are available regardless of posture.

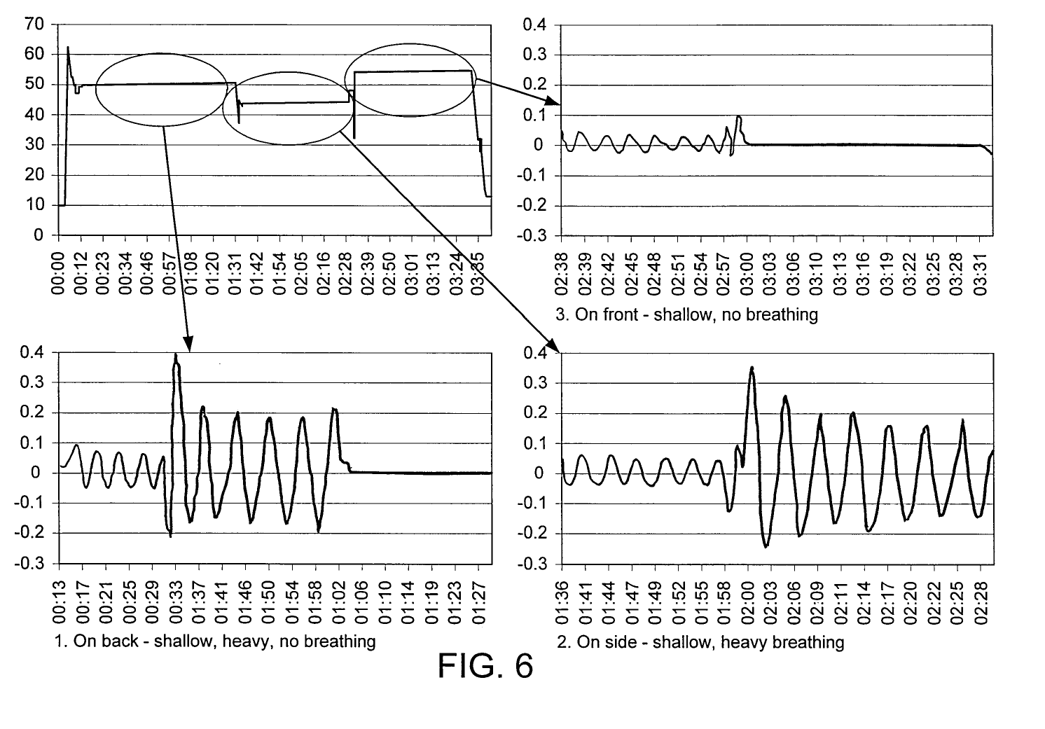

Turning now to FIG. 6, the graph of FIG. 5 is repeated to a smaller scale at the top left hand corner. Three zones of that graph are ballooned and then repeated, as indicated by their arrows, at an enlarged scale indicating the separate analysis of the three postures previously discussed. The plot labelled 1 shows the breathing pattern of a subject lying on the back and is breathing in a shallow manner, then breathing deeply and finally not breathing at all. This plot shows characteristic patterns of low amplitude (shallow breathing), large amplitude (heavy breathing), and a zero amplitude (no breathing). Any or all of these patterns can be specifically monitored to assess the patient's condition.

The plot labelled 2 shows the breathing pattern of a subject lying on the front, that is shallow breathing followed by no breathing.

The plot labelled 3 shows the breathing pattern of a subject lying on his side, that is shallow breathing followed by heavy breathing. The respective patterns are sufficiently distinguishable that the breathing condition and posture of the subject can be determined.

The invention therefore provides a valuable tool to assist the monitoring of subjects with a view to determining breathing patterns and/or breathing conditions. This can be of immense value in a medical field, for example for monitoring sleep apnoea. The invention can also be used with immense advantage in the context of infant monitoring (cot death), animal welfare, patient monitoring, prisoner surveillance and so on. In certain applications the display monitor 17 may be sufficient. However, an alarm may be provided to warn of the detection of a potentially dangerous body function, or subject movement.

This invention is of particular for use in care homes for the elderly as it enables 24 hour surveillance of a plurality of patients by a single nurse. It is becoming increasingly common that patients or their relatives will seek to make claims in respect of alleged neglect. The memory 18 provided by this invention provides a record of all movements of a patient as well as the onset of any condition.

1. A body function sensor (1, 2, 3) arranged to detect a body function of an animal body by detecting any phase difference between electromagnetic energy to which the animal body is exposed and electromagnetic energy received by an analyser means, characterised in that the sensor is arranged to create a low frequency (LF) electromagnetic field at a location where the animal body is to be positioned and to detect any phase changes in this electromagnetic field, a control means to adjust the frequency of the electromagnetic field until it is resonant, and the analyser means is arranged to assess minor phase changes in the electromagnetic field as an indication of the body function.

2. A body function sensor as claimed in claim 1, characterised in that the analyser means is arranged to determine the presence or movement of an animal body at the location by detecting a change in resonant frequency caused by the presence or movement of the animal body.

3. A body function sensor as claimed in claim 1, characterised in that the analyser means is arranged to determine at least one body function of the animal body by detecting changes in the phase of the electromagnetic field caused by the movement of the animal body at the location.

4. A body function sensor as claimed in claim 1, characterised in that a variable frequency waveform generator has its frequency controlled by the control means and is arranged to transmit its output through a transmit means to an antenna for transmitting the low frequency (LF) electromagnetic field at the location.

5. A body function sensor as claimed in claim 4, characterised in that the antenna includes a single conductor positioned at the location and also serving to transmit a received signal to an analyser means.

6. A body function sensor as claimed in claim 4, characterised in that a receive antenna is provided to receive the electromagnetic field and to convey it to the analysis means.

7. A body function sensor as claimed in claim 4, characterised in that the antenna (4) and the receive antenna include separate conductors positioned at the location.

8. A body function sensor as claimed in claim 7, characterised in that the separate conductors are intercalated.

9. A body function sensor as claimed in claim 4, characterised in that the variable frequency waveform generator includes a source of LF radio-frequency radiation.

10. A body function sensor as claimed in claim 4, characterised in that the variable frequency waveform generator is connected to the transmit antenna.

11. A body function sensor as claimed in claim 1, characterised in that the analyser means includes an Analogue/Digital converter to receive a signal representative of the electromagnetic field, and a Fast Fourier Transformer, or its electronic equivalent, to determine the difference in phase between the transmitted LF signal and a received LF signal changed by said body function.

12. A body function sensor as claimed in claim 11, characterised in that the analyser means includes a filter to filter the difference in phase, and a monitor/display means operated by the control means to monitor/display the results of the sensor.

13. A body function sensor as claimed in claim 1, characterised in that a memory is provided to record the history of phase changes.

14. A method of monitoring a body function of an animal body at a location, characterised by creating a low frequency electromagnetic field at the location, adjusting the frequency of the electromagnetic field until it is resonant, assessing phase changes in the electromagnetic field, and analysing such phase changes to monitor the body function.

15. A method as claimed in claim 14, characterised by using a change in the frequency to indicate the presence or absence of the animal body at the location.

16. A method as claimed in claim 15, and in the case where the animal body is a human being, characterised by using either a bed or a chair as the location.

17. A method as claimed in claim 16, characterised by recording changes in the frequency to provide a history of the times when the human being was in the bed or the chair.

18. A method as claimed in claim 16, characterised by using the analysis of the phase changes to provide a real-time assessment of the well-being of the human being.

19. A method as claimed in claim 14, characterised by recording phase changes to provide a history of the monitored body function.

20. A method as claimed in claim 16, characterised by operating an alarm whenever the human being is absent from the bed or chair, or the well-being of the human being is compromised.
